(19) United States
(12) Patent Application Publication
(10) Pub. No.: US 2010/0113453 A1
(43) Pub. Date: May 6, 2010

(54) Sublingual Formulations of D-Cycloserine and Methods of Using Same

(76) Inventors: Jason P. McDevitt, Williamsburg, VA (US); Michael Davis, Stone Mountain, GA (US); Kerry J. Ressler, Atlanta, GA (US)

Correspondence Address: JASON P. MCDEVITT, 124 COUNTRY CLUB DRIVE, WILLIAMSBURG, VA, 23188, US

(21) Appl. No.: 12/594,371

(22) PCT Filed: Apr. 10, 2008
(86) PCT No.: PCT/US08/59876

§ 371 Date: Oct. 2, 2009

Related U.S. Application Data
(60) Provisional application No. 60/910,913, filed on Apr. 10, 2007.

(51) Int. Cl. A61K 31/42; C07D 261/04; A61P 25/00; A61K 31/519; A61K 31/53; A61K 31/4985

(52) U.S. Cl. 514/243; 548/244; 514/380; 514/252.16; 514/250; 514/262.1

(57) ABSTRACT

The invention provides methods and compositions for treating anxiety-related disorders in a subject. The methods include sublingually administering D-cycloserine to a subject with the anxiety-related disorder, either alone or in combination with extinction training.

### CROSS-REFERENCE TO RELATED APPLICATIONS

This application claims the benefit of and priority to U.S. Provisional Patent Application Ser. No. 60/910,913, filed Apr. 10, 2007, the entire disclosure of which is incorporated by reference herein.

### FIELD OF INVENTION

The invention relates generally to compositions and methods for the treatment of anxiety disorders, and more specifically to compositions suitable for sublingual administration of D-cycloserine (DCS) and to methods of treating anxiety disorders that include the sublingual administration of DCS.

### BACKGROUND

Classical fear conditioning occurs when an effectively neutral stimulus is paired with a noxious aversive stimulus (unconditioned stimulus [US]), such as, footshock. Afterwards, the previously neutral stimulus (i.e., now the conditioned stimulus [CS]) is able to elicit a variety of autonomic, hormonal, and skeletal responses that accompany the conscious experience of fear in humans. These responses can be used to operationally define fear in laboratory animals. The fear-eliciting properties of the CS can be extinguished by repeatedly presenting the CS in the absence of the US. It is believed that such extinction does not reflect unlearning of the original association, but instead involves the formation of new associations that compete with the previously conditioned response.

A reduced ability to extinguish high-anxiety responses resulting from fear memories is a significant clinical problem for a wide range of anxiety disorders including specific phobias, social phobias, panic disorder, obsessive-compulsive disorder (OCD), generalized anxiety disorder, and post-traumatic stress disorder (PTSD). These disorders are characterized by a high-anxiety response to a stimulus that is disproportionate to the threat. Treatment for these disorders often relies upon the progressive extinction of the high-anxiety response to the stimulus. Hence, a pharmacological enhancement of extinction would likely be of considerable clinical benefit in these conditions.

DCS has long been used as an antibiotic to treat tuberculosis. Human clinical studies, pairing administration of DCS with psychotherapy for treatment of various anxiety disorders, including specific phobias, social anxiety disorders, panic disorders, and obsessive-compulsive disorders, has also demonstrated that DCS facilitates extinction and improves subject response to therapy, for example, psychotherapy.

In these studies, DCS routinely is administered to subjects prior to psychotherapy sessions, typically one to three hours prior to therapy. Subjects instructed to take DCS one to three hours prior to psychotherapy sessions are typically expected to self-administer the drug at the appropriate time. Delaying administration of the drug until the commencement of the psychotherapy session can reduce therapeutic efficacy, as it takes time for the drug to be absorbed sufficiently to reach therapeutically active levels in the body. However, the likelihood that a subject will forget, self-administer too early, or otherwise fail to comply to self-administer prescribed DCS medication at the appropriate time is not insignificant, and potentially reduces efficacy of the DCS.

In some instances, subjects have difficulty swallowing pills. Globus hystericus, a form of conversion disorder, is characterized by an uncomfortable sensation of a mass in the esophagus or airway. Anxiety or psychological conflict correlates with the onset and progression of the sensation. Furthermore, a small but significant number of people have a fear of swallowing pills (“phagophobia”), and incidence is disproportionately high for individuals afflicted with other anxiety disorders. Accordingly, simply having an alternative formulation for administration of D-cycloserine is, in and of itself, advantageous, since a sublingual formulation provides a route of administration of D-cycloserine that does not require active swallowing of a tablet, capsule, or other common oral pharmaceutical dosage form.

Accordingly, there is a need for improved methods and DCS compositions that achieve effective serum concentrations in a subject so that the effective concentration of DCS substantially coincides with or assists in the consolidation of the effects of extinction training.

### BRIEF SUMMARY OF THE INVENTION

In one aspect, the invention provides a pharmaceutical composition suitable for sublingual administration comprising about 10 mg to about 80 mg D-cycloserine, wherein upon administration to a patient, the patient achieves a therapeutically effective serum concentration of D-cycloserine within 1 hour of administration, within forty minutes of administration, within thirty minutes of administration, or even within twenty minutes of administration.

The pharmaceutical compositions can further comprise at least one pH-adjusting substance, and/or at least one of: a glidant, a lubricant, a binder, a sweetener, a flavoring, and a coloring agent, and/or least one effervescent agent.

The pharmaceutical compositions can be in the form of a solid, for example, a tablet, capsule, wafer or gel. Alternatively, the pharmaceutical compositions can be in the form of a spray.

In another aspect, the invention provides a method for treating an anxiety-related disorder in a subject in need thereof. The method comprises administering sublingually a therapeutically effective amount of D-cycloserine such that the patient achieves a therapeutically effective serum concentration of D-cycloserine within two hours, within one hour, within forty minutes, or within thirty minutes or less of DCS administration.

Such methods can further comprise administering extinction training, for example, administered within two hours, within one hour, or within one-half hour of administering D-cycloserine. In some embodiments, the D-cycloserine is administered before, after or substantially simultaneously with the administration of extinction training.

The methods can include treating an anxiety disorder, a mood disorder, an addictive disorder, an eating disorder, a movement disorder, erectile dysfunction, insomnia, chronic pain. The method of treating erectile dysfunction can further include administering a phosphodiesterase-5 (PDE-5) inhibitor, for example, sildenafil citrate.

In another aspect, the invention provides an improved method of treating an anxiety-related disorder by extinction training in a subject in need thereof. The improvement comprises sublingually administering a therapeutically effective amount of D-cycloserine to the subject before or substantially simultaneously with the extinction training.

### DETAILED DESCRIPTION OF THE INVENTION

The present invention is directed to formulations of D-cycloserine, and methods and compositions for treating anxiety-related disorders. Described herein are methods and compositions for treatment of anxiety-related disorders using sublingual formulations of D-cycloserine. Such methods can be used in conjunction with and/or augment psychotherapy (or related treatments) for treatment of anxiety-related disorders, for example, anxiety disorders, eating disorders, movement disorders, mood disorders, addictive disorders, erectile dysfunction, and sleep disorders, such as, insomnia.

The compositions and methods disclosed herein may be used in connection with extinction training, for example, with psychotherapy. The serum levels of DCS in a subject at the time of such an extinction learning event should be sufficiently high to be therapeutically effective, and these serum levels can be achieved using the sublingual compositions comprising DCS disclosed herein.

D-cycloserine, or DCS, refers to the chemical D-cycloserine (4-amino-1,2-oxazolidin-3-one; CAS Registry No. 68-41-7), or pharmaceutically acceptable salts thereof. DCS is an FDA (United States Food and Drug Administration)-approved drug for treatment of tuberculosis, and is sold by Eli Lilly and Company under the trade name Seromycin®. DCS is a structural analog of D-alanine, and is a broad-spectrum antibiotic produced by some strains of Streptomyces orchidaceus and S. garphalus. DCS has antibiotic activity in vitro against growth phase Gram-negative bacteria such as Escherichia coli, some strains of Staphylococcus aureus, and Chlamydia species, among others. The minimum inhibitory concentrations (MIC) in vitro for typical Mycobacterium tuberculosis strains range from about 6-25 μg/mL.

For the treatment of tuberculosis, DCS is generally dosed at 500-1000 mg/day divided twice daily (PDR 1997) with chronic treatment. At a dose of 500 mg/day, serum concentrations of 25-30 μg/ml are generally maintained. Administration of, e.g. known oral tablets typically result in peak serum concentrations occurring within 3-8 hours after dosing-with primarily renal excretion with a half-life of 10 hours.

At these typical doses for the treatment of tuberculosis, DCS can give rise to significant neurological side effects in treated subjects. Typical side effects on chronic dosing schedules (who were generally chronically ill with tuberculosis) include drowsiness, depression, headache, confusion, tremor, vertigo, and memory difficulties, paresthesias, and seizure.

As contemplated herein, a therapeutically effective amount of DCS is lower than the amounts typically used for the treatment of tuberculosis. A “therapeutically effective amount” or “therapeutically effective dose” of the pharmacologic agent is an amount of the pharmacologic agent that typically results in an improved treatment, for example, a greater therapeutic benefit, or a therapeutic benefit for a longer time, relative to that observed in the absence of administering the pharmacologic agent.

In some embodiments, a therapeutically effective amount of DCS used in the disclosed methods of treating depression relates to a sub-antimicrobial dose of DCS. A sub-antimicrobial dose refers to a dose of DCS that is less than or equal to 2 mg DCS per kg body weight of the subject (i.e., less than or equal to 2 mg/kg), and greater than about 0.2 mg/kg (this lower limit is needed to maintain efficacy for facilitation of extinction). When administered to a subject, sub-antimicrobial doses of DCS achieve peak serum concentrations in the subject of less than or equal to about 5 μg/mL, although there is substantial variability between subjects. At these low concentrations of DCS, the drug no longer kills most microorganisms, including those that are ordinarily susceptible to higher DCS concentrations typically reached in the body when DCS is used to treat tuberculosis (i.e., 500 mg or 1000 mg per day).

When administered to a subject, the compositions disclosed herein achieve a therapeutically effective serum concentration within, for example, 15 minutes, 20 minutes, 30 minutes, 40 minutes, 1 hour, or even 2 hours after administration. Peak serum concentrations may be obtained, upon administration of the disclosed fast-acting compositions with for example, one hour, one and one-half hours, two hours, or three hours after administration.

In certain circumstances, the sublingual formulations disclosed herein are absorbed rapidly in the body permitting administration at, for example, a therapist's office, with no need to significantly delay the start of the therapy session to accommodate the pharmacokinetics of the drug. Such sublingual formulations can provide a distinct advantage relative to conventional oral formulations, reducing the therapeutic importance of a subject remembering to take the drug at the required time, and thereby maintaining the full benefits of DCS administration for subjects for whom compliance might otherwise be imperfect. Such sublingual formulations may also provide a distinct advantage relative to conventional formulations for oral administration for subjects who have difficulty swallowing conventional oral dosage formulations.

The compositions include, but are not limited to, sprays, foams, films, pastes, gels, tablets, capsules, wafers, and patches. In some embodiments, the sublingual formulation comprises DCS and an additional active agent useful for the treatment of an anxiety-related disorder. For example, in one embodiment of the invention, the pharmaceutical composition is a sublingual formulation comprising DCS and sildenafil citrate.

Serum concentration levels of DCS in subjects administered DCS are a function of numerous factors, including body weight, metabolism, and the amount of drug ingested. The timing of administration and the therapeutically effective dose of DCS in a given subject will depend on the severity of symptoms, in addition to the age, sex, and size of the subject being treated, among other variables. Preferably the serum concentration of DCS in a subject will be at least 1 μg/mL at the time of extinction training.

All microorganisms do not have the same susceptibility to DCS. Accordingly, while it is possible that a sub-antimicrobial dose of DCS can still kill a small subset of microorganisms, sub-antimicrobial doses of DCS generally will not have a significant antimicrobial effect in the body. When administered to adult human subjects, a sub-antimicrobial dose of DCS generally comprises a drug formulation (e.g., pill, capsule, tablet) of DCS containing DCS in an amount equal to or less than 100 mg, preferably less than 80 mg DCS (e.g., about 5 mg to about 100 mg, or about 10 mg to about 100 mg, or even about 10 mg to about 80 mg) to provide a greater margin between the concentration of DCS and the minimum inhibitory concentration (MIC) of DCS against microorganisms active in a subject's body.

Children being administered DCS for treatment of tuberculosis are normally dosed at a level between about 10-20 mg/kg. A sub-antimicrobial dose of DCS, when administered to a child subject according to the methods of the present invention, comprises less than or equal to 2 mg/kg, and generally achieves peak serum concentrations of DCS in the child subject of less than or equal to 5 μg/mL.

Because the dosages of DCS contemplated herein are lower than those used to treat tuberculosis, the side effects of DCS will generally be greatly reduced by infrequent dosing at concentrations five to twenty-fold lower than those routinely used for TB treatment.

In some embodiments, DCS is administered on an acute basis. Acute administration of a pharmacological agent to a subject generally refers to a single exposure of the subject or patient to a therapeutically effective amount of the pharmacological agent within an extended time period, i.e. a time period of three days or longer. For example, a once- or twice-weekly administration of DCS constitutes acute administration.

It is contemplated that other agents may be also administered before, during, or after the administration of extinction training and/or DCS. For example, a B vitamin, such as one or more of the B-complex vitamins, can be administered to a subject in addition to DCS, as part of the contemplated methods. A B-complex vitamin includes thiamine (B1), riboflavin (B2), niacin (B3), pyridoxine (B6), folic acid (B9), cyanocobalamin (B12), pantothenic acid and biotin. For example, pyridoxine can be additionally administered at up to ten times the dosage of DCS. In an embodiment, the DCS for administeriation as contemplated herein is provided in a composition that includes a B-complex vitamin, e.g. pyridoxine, for example, as a tablet or other composition that includes for example 50 mg DCS and 50 mg pyridoxine. DCS has been reported to reduce the levels of certain important chemicals in the blood of subjects, including calcium, folic acid, magnesium, vitamin K, and vitamin B6 and vitamin B12.

Administration of DCS and an additional active agent is contemplated, e.g., administration of another anxiety related disorder agent, e.g., sildenafil, or e.g., administration of erectile dysfunction agents, e.g., a phosphodiesterase-5 inhibitor.

### Methods

The invention provides methods of treating anxiety-related disorders in a subject in need thereof. The method comprises administering sublingually a therapeutically effective amount of D-cycloserine such that the patient achieves a therapeutically effective serum concentration of DCS within, e.g., two hours, within one hour, within 40 minutes, within 30 minutes, or even within 15 minutes of administration. Such methods optionally may also include administration of extinction training.

In some embodiments, the disclosed methods include administering DCS and extinction training. The disclosed sublingual formulations of DCS may be administered within about two hours of the commencement or termination of an extinction training event, e.g., within about 0 to about 2 hours, within about ½ to 1 hour, or within about ½ to 2 hours. DCS may be administered to a subject before or after the subject experiences an extinction learning event. Alternatively, a disclosed sublingual DCS formulation may be administered substantially at the same time as an extinction learning event.

Anxiety-related disorders relate to those disorders characterized by fear, anxiety, addiction, and the like. Patients with anxiety-related disorders can have a single such disorder, or may have a constellation of disorders. The anxiety-related disorders contemplated in the present invention include, but are not limited to, anxiety disorders, addictive disorders including substance-abuse disorders, mood disorders (e.g., depression and/or bipolar disorder), movement disorders such as Tourette's syndrome, erectile dysfunction (impotence resulting from a man's inability to obtain or maintain an erection of his penis), insomnia (e.g., chronic insomnia), and eating disorders (e.g., anorexia).

In an embodiment, the method provides a treatment of a male subject afflicted with erectile dysfunction. The method comprises administering to a subject a sublingual formulation comprising DCS and a phosphodiesterase-5 (“PDE-5”) inhibitor within, for example, about 30 minutes, or one hour or two hours, prior to a sexual encounter.

Anxiety disorders include, but are not limited to, panic disorder, agoraphobia, social phobia, specific phobia, post-traumatic stress disorder (PTSD), obsessive-compulsive disorder, and generalized anxiety disorder. The disorders contemplated herein are defined in, for example, the DSM-IV (Diagnostic and Statistical Manual of Mental Disorders (4th ed., American Psychiatric Association, Washington D.C., 1994)).

Contemplated methods may be used to treat subjects that experience a deleterious, high-anxiety response to a given stimulus. Such a response is characterized by a high level of anxiety that is disproportionate to the threat represented by the stimulus. Accordingly, a stimulus that generates little if any anxiety in most subjects would generate substantial anxiety in a subject undergoing a deleterious, high-anxiety response. These deleterious, high-anxiety responses cause or exacerbate symptoms characteristic of the medical disorders described herein.

Insomnia is an anxiety-related disorder that can be defined as the inability to fall asleep or to stay asleep for a sufficient amount of time during regular sleeping hours. It includes acute insomnia, which occurs in either a transient or short term form, and chronic insomnia. It also includes initial insomnia, defined as difficulty in falling asleep; middle insomnia, defined as awakening in the middle of the night followed by eventually falling back to sleep, but with difficulty; and terminal insomnia, defined as awakening before one's usual waking time and being unable to return to sleep.

Also contemplated herein are methods for treating pain, e.g., chronic pain (i.e., pain that has lasted more than three months) in a subject in need thereof that includes administering sublingually a therapeutically effective amount of D-cycloserine such that the patient achieves a therapeutically effective serum concentration within, e.g., two hours, within one hour, within 40 minutes, within 30 minutes, or even within 20 minutes of administration. Such methods may also include administration of extinction training. Chronic pain in a patient can result in significant psychological and emotional effects and can, for example, limit a person's ability to fully function.

The goal of extinction training is to pair a stimulus that previously provoked a deleterious, high-anxiety response with a new learning that the stimulus will not lead to a negative outcome, thereby generating in a subject a new, more appropriate response to the stimulus to compete with and ideally replace the previous disproportionate response. Extinction training refers to exposure to a stimulus or situation in the absence of an aversive consequence. For example, a subject having a deleterious, high anxiety response to a given stimulus or situation is exposed to the stimulus or situation in the absence of an aversive consequence. A typical goal of extinction training is to produce new learning in the subject that results from the pairing of the original stimulus (e.g., fear stimulus) or situation with a non-deleterious outcome, thereby generating, in subsequent exposures to the stimulus, a more appropriate response in place of a deleterious, high anxiety response. An extinction learning event refers to a completed stimulus/response extinction training cycle.

One form of extinction training entails psychotherapy. For example, the methods of the invention contemplate treatment of anxiety disorders by: (i) administering a sublingual formulation of DCS to a subject; and (ii) exposing the subject to psychotherapy to treat an anxiety-related disorder. Suitable methods of psychotherapy include exposure-based psychotherapy, cognitive psychotherapy, and psychodynamically oriented psychotherapy. For example, the methods disclosed herein contemplate treatment for anxiety-related disorders in a subject may include (i) waiting for a subject to arrive at the office of, e.g., a therapist, (ii) administering to the subject a sublingual formulation of DCS, and (iii) conducting a psychotherapy session with the subject.

The disclosed methods and compositions are therapeutically viable for therapists to store and dispense the drug to patients as appropriate. This embodiment eliminates the requirement for patients to obtain and then fill a prescription, and reduces waste based on over-projections of the number of therapy sessions.

One method of psychotherapy that is specifically contemplated is the use of virtual reality (VR) exposure therapy to treat an anxiety disorder using the methods of the invention. Another method of psychotherapy that is particularly beneficial when utilized in accordance with the methods and compositions of the present invention is cognitive behavioral therapy (“CBT”). CBT is a form of psychotherapy that combines cognitive therapy and behavior therapy, and emphasizes the critical role of thinking in causing people to act and feel as they do. Therefore, if an individual is experiencing unwanted feelings and behaviors, CBT teaches that it is important to identify the thinking that is causing the undesirable feelings and/or behaviors and to learn how to replace this deleterious thinking with thoughts that lead to more desirable reactions.

In one embodiment, subjects suffering from social phobia undergo weekly cognitive behavioral therapy sessions to treat the affliction. At the outset of each session, subjects are administered a sublingual formulation of DCS (e.g., 40 mg). Relative to subjects treated only via cognitive behavioral therapy, or to subjects treated via cognitive behavioral therapy and a placebo, anxiety associated with social phobia is expected to be reduced to a greater extent in subjects treated with a combination of cognitive behavioral therapy and acute doses of DCS. In a variation on this embodiment, the subjects are administered DCS after cognitive behavioral therapy.

In another embodiment, DCS can be administered after extinction training, for example, the subjects are administered DCS after cognitive behavioral therapy only if the cognitive behavioral therapy yields positive results on that day, as determined by the subject and/or therapist. The benefits of administering DCS after such extinction training can be inversely correlated with the time elapsed between such training and the time which DCS reaches therapeutically effective levels within the body. In such embodiments, DCS should be administered as quickly as possible.

Another form of extinction training is provided by biofeedback, which is particularly useful in enabling subjects to learn to control physiological processes that normally occur involuntarily, such as blood pressure, heart rate, muscle tension, and skin temperature. As used herein, “biofeedback” refers to a technique in which subjects are trained to improve their health by using signals from their own bodies to control their own physiological responses.

In one embodiment, a subject suffering from chronic pain undergoes biofeedback sessions to help alleviate the pain. Upon conclusion of each session wherein the subject has made progress in learning/developing responses that reduce the chronic pain, the subject is administered a sublingual formulation of DCS (e.g., 50 mg) in order to consolidate the desired learning.

In another embodiment, a subject suffering from phantom limb syndrome undergoes thermal biofeedback sessions to reduce and hopefully eliminate the symptoms. At the start of each session, the subject is administered a sublingual formulation of DCS (40 mg). The acute DCS treatment can provide benefits to the subject beyond those that would be obtained if the subject underwent biofeedback without supplemental DCS.

Another form of extinction training can be provided by pharmacotherapy. For example, a man afflicted with erectile dysfunction can have an extinction learning event based on a positive sexual outcome achieved with the assistance of a PDE-5 inhibitor, such as, sildenafil, tadalafil, vardenafil, and/or udenafil. By administering a sublingual formulation of DCS and sildenafil, e.g., sildenafil citrate, to a subject with erectile dysfunction, followed by a successful sexual outcome, the heightened confidence and reduced sexual performance anxiety resulting from a successful outcome can be consolidated in the subject's psyche, thereby facilitating extinction of any deleterious performance anxiety associated with sexual intercourse.

Extinction training does not always require intervention of a trained specialist. Individuals can carry out extinction training on themselves.

### Formulations

The pharmaceutical compositions contemplated by this invention can be formulated and administered sublingually to a subject as described below.

The invention encompasses the preparation and use of pharmaceutical compositions comprising DCS as an active ingredient useful, for example, in the disclosed methods. Such pharmaceutical compositions may consist of DCS alone or the pharmaceutical composition may comprise DCS and, for example, one or more pharmaceutically acceptable carriers, one or more additional active ingredients, or a combination of the foregoing. The active ingredient(s) may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable ester or salt” means an ester or salt form of the active ingredient which is compatible with any other ingredients of the pharmaceutical composition, which is not deleterious to the subject to which the composition is to be administered.

The formulations of the invention may conveniently be presented in unit dosage form and may be prepared by any methods well known in the pharmacy arts. The amount of the composition that may be combined with a carrier material to produce a single dose may vary depending upon the subject being treated. The relative amounts of the active ingredient, the pharmaceutically acceptable carrier, and any additional ingredients in a pharmaceutical composition of the invention may vary, depending upon the identity, size, and condition of the subject treated. By way of example, the composition may comprise between 0.1% and 100% (w/w) active ingredient.

Sublingual administration refers to therapeutic administration of a pharmaceutical composition under the tongue. The pharmaceutical compositions may be formulated so they dissolve slowly, moderately quickly, or rapidly. Sublingual compositions also include those formulations administered under the tongue, for example, by spraying and those that constitute a medical device that comprise a therapeutic agent and is removed once the agent has been substantially released or dissolved.

Under certain circumstances, a sublingual formulation of DCS permits the use of lower doses of DCS than would be required for typical oral formulations for the same indication since a greater portion of the medication is absorbed directly into the blood stream. For example, the present invention contemplates about a 10-50% reduction in dose relative to effective doses of DCS tablets useful for the same indication. Fast acting compositions may comprise between about 25 mg and about 80 mg, between about 15 mg to 50 mg, or even about 10 mg to about 30 mg DSC.

Furthermore, under certain circumstances, relative to oral routes of administration (i.e., administered by mouth and swallowed), sublingual administration of DCS allows for rapid absorbance, and hence rapid onset of effect. Relative to nasal and pulmonary delivery formulations, sublingual formulations are discreet, easy, and convenient, and can exhibit more predictable pharmacokinetics.

Sublingual administration of a DCS may take numerous forms. In one embodiment, it is contemplated that DCS can be in the form of a tablet or packed powder. The sublingual administration of DCS may comprise a medical device, such as, a patch. The patch may be placed under the tongue. The patch may have adhesive qualities to prevent the movement, loss or swallowing of the patch. The patch may be ingestible in case of accidental swallowing or to allow for easy disposal of the patch. In another embodiment the patch may be removed from under the tongue after the prescribed time. In yet another embodiment, the sublingual administration of DCS can take the form of a paste, gel, or film. The paste, gel, or film would be applied under the tongue. The viscosity of the paste, gel, or film can be adjusted to allow for the retention under the tongue. In another embodiment, it is contemplated that the present invention can be a liquid. It is further contemplated that the liquid can be in the form of a spray or drops.

It is contemplated that the sublingual administration of DCS can be made with a formulation specifically formulated for particularly rapid dissolution. For example, such a formulation may be a hard, compressed, rapidly-dissolving tablet adapted for direct sublingual dosing that may include particles comprising an active ingredient and a protective material. Such a tablet may also include a matrix made from a non-direct compression filler, a wicking agent, and/or a hydrophobic lubricant. The tablet matrix may comprise at least about 60% rapidly water-soluble ingredients based on the total weight of the matrix material and/or may have a hardness of between about 15 and about 50 Newtons, a friability of less than 2% when measured by U.S.P., and/or may be adapted to dissolve in the mouth of a patient in less than about 60 seconds and thereby liberate the particles.

In an embodiment, the formulations of the invention can include a pH-adjusting agent, which alters or adjusts the pH of the sublingual area upon dissolution.

Contemplated formulations for sublingual administration include solid pharmaceutical compositions that rapidly disintegrate in the mouth of a subject, upon placement under the tongue. Rapid disintegration means that the pharmaceutical composition is disintegrated within 30 seconds in water at 37° C., as measured according to the procedure described in Remington's Pharmaceutical Sciences, 18th Edition (Ed. A. R. Genaro), 1990, pp 1640-1641; see also US Pharmacopeia, Chapter <701>.

In an embodiment, the pharmaceutical compositions of the invention are tablets or lozenges which comprise a rapidly disintegrating composition of a pharmaceutically acceptable water-soluble or water-dispersable carrier material. Tablets and lozenges comprising a rapidly disintegrating composition of a pharmaceutically acceptable water-soluble or water-dispersable carrier material are known in the art.

A sublingual DCS tablet can comprise effervescent agents, i.e., agents that evolve gas, for example, by means of a chemical reaction that takes place upon exposure to an aqueous solution such as water or saliva. Effervescent agents allow enhanced adsorption of the active ingredient across the mucosal membranes in the sublingual cavity.

Sublingual formulations may include mucosal membrane transport enhancing agents, for example, peppermint oil, spearmint oil, menthol, pepper oil, eucalyptus oil, cinnamon oil, ginger oil, fennel oil, and dill oil, hydrochloric acid, phosphoric acid, acetic acid, citric acid, lactic acid, oleic acid, linoleic acid, lauric acid, palmitic acid, benzoic acid, and salicylic acid.

In another embodiment, the present invention contemplates a sublingual composition comprising a solid preparation comprising granules wherein each granule comprises a fine particulate core and a drug layer comprising DCS coated on the fine particulate core, for example, as disclosed in U.S. Pat. No. 5,624,687.

In yet another embodiment of the present invention, it is contemplated that the sublingual composition comprises an ordered mixture of one or more bioadhesive carrier substances coated with the pharmaceutically active agent or agents in a fine particulate form, as disclosed in PCT application Publication No. WO 00/16750 and EP 0324725. Such a composition may include DCS in a finely dispersed state which substantially covers the surface of substantially larger carrier particles. Such compositions disintegrate rapidly in water, thereby dispersing their contents of microscopic drug particles.

Contemplated formulations can also include a mucoadhesion-promoting agent. A mucoadhesion-promoting agent is a substance that aids in the adhesion of a compound to a biological or mucosal surface. The mucoadhesion-promoting agent may possess properties to swell and expand in contact with water, and thus, for example, facilitate tablet or carrier particles disintegration when wetted with saliva.

The mucoadhesion-promoting agent can be, for example, a polymeric substance, for example, a substance with an average molecular weight above 5,000 (weight average). The level of hydration of the mucoadhesion-promoting agent interface may be of importance in the development of mucoadhesive forces. A fast-swelling polymer may quickly initiate mucoadhesion. Additionally, it is contemplated that the hydration of mucoadhesive compounds also makes them useful as absorption enhancers.

The mucoadhesion-promoting agent may have a particle size between about 1 μmol and about 100 μmol. When the particles of this agent are mixed with the carrier particles to form an ordered mixture, their size falls within the lower part of the size interval, and suitably their size is then below 10 μmol. When the mucoadhesion-promoting agent is incorporated in the carrier particles, its particle size may be within the upper part of the size interval.

A variety of polymers known in the art can be used as mucoadhesion-promoting agents. An example of a contemplated polymer is one that is swellable while also being substantially insoluble in water. In one embodiment, the swelling factor by volume when brought into contact with water or saliva should preferably be, for example, at least 10 to 20. Examples of such mucoadhesion-promoting agents include cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose and sodium carboxymethyl cellulose (NaCMC); starch derivatives such as moderately cross-linked starch, acrylic polymers such as carbomer and its derivatives (Polycarbophyl, CarbopolQ, etc.); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); microcrystalline cellulose (Avicela); and crosscaramellose. It is also contemplated that combinations of two or more mucoadhesive polymers be used. More generally, any physiologically acceptable agent showing mucoadhesive characteristics may be used successfully to be incorporated in the carrier. Mucoadhesiveness can be determined in vitro, for example, according to G. Sala, et al., (Proceed. Int. Symp. Contr. Release. Bioact. Mat. 16:420, 1989).

Depending on the type and the proportion of the mucoadhesion-promoting agent used, the rate and intensity of mucoadhesion may be varied. According to one embodiment of the invention, substances with high and rapid capacity for swelling are preferred.

In some embodiments, formulations that include mucoadhesion-promoting agents additionally comprise carrier particles. The mucoadhesion-promoting agent can be present on the surface of the carrier particles, but it may optionally also be present within these particles. It is contemplated that the carrier particles contain, for example, from about 0.1 up to 25 weight percent of mucoadhesion-promoting compound, based on the total composition. The mucoadhesion-promoting agent content may be from about 1.0 to 15.0 weight percent. The carrier used may comprise any substance which is pharmaceutically acceptable, is highly soluble in water, and which can be formulated into particles fit for incorporating a mucoadhesion-promoting agent. A number of such substances are known to the person skilled in this art. For example, sugar, mannitol and lactose, or pharmaceutically acceptable inorganic salts, such as, sodium chloride or calcium phosphate may be used.

In another embodiment, it is contemplated that the carrier also comprises a fragmentation-promoting agent. A fragmentation-promoting agent is a brittle material which is readily crushed or broken up when a pharmaceutical composition of which it forms a part is compacted into tablets. For example, and without limiting the present invention to any particular theory, if a mucoadhesion-promoting agent also is incorporated within the carrier as well as being added to the carrier surface, further surfaces of mucoadhesion-promoting agent may then be exposed for hydration. This effect is especially pronounced when the mucoadhesion-promoting agent also serves as a disintegrant. As examples, mannitol and lactose have been found to be particularly suitable as fragmentation-promoting agents.

In another embodiment, it is contemplated that a pharmaceutically acceptable surfactant is added to a composition. Although the present invention is not limited to any particular mechanism, it is believed that the increased wetting effect of the surfactant enhances the hydration of the composition, e.g., hydration of carrier particles, which results in faster initiation of mucoadhesion. In one embodiment, the surfactant is in a finely dispersed form and well mixed with the active agent or agents. The amount of surfactant should be, for example, from about 0.5 to 5.0 weight percent of the composition, and preferably from about 0.5 to 3.0 weight percent. As examples of suitable surfactants may be mentioned sodium lauryl sulfate, polysorbates, bile acid salts and mixtures of these.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

### EXAMPLES

The example that follows is intended in no way to limit the scope of this invention but is provided to illustrate the methods present invention. Many other embodiments of this invention will be apparent to one skilled in the art.

### Example 1

### Comparison of Absorption into the Blood Following Sublingual Versus Oral Administration of D-Cycloserine

This example demonstrates that D-cycloserine can be administered sublingually to provide therapeutically effective amounts of D-cycloserine in the blood.

Subjects: 18 male, Sprague-Dawley rats (Charles River, Raleigh N.C.).

Procedure: The rats were implanted with intravenous catheters using the methods of Thrivikraman et al., (“Jugular vein catheterization for repeated blood sampling in the unrestrained conscious rat” (2002) Brain Res. Brain Res. Protoc., 10(2), p. 84-94). Following two days of recovery, rats were lightly anesthetized with isofluorothane and given DCS either sublingually (n=10) or orally (n=8). D-cycloserine was dissolved in saline and given at a volume of 75 μL/rat sublingually and (after dilution by a factor of two) 150 μL/rat when administered orally (concentration of 15 mg/kg). Aliquots of 0.5-1.0 mL blood samples were collected: at baseline before DCS administration; and subsequently at 10, 20, 40, and 80 minutes after DCS administration. Blood samples were injected into pre-heparinized tubes and immediately placed on ice. Because the assays required at least 1 mL of plasma, samples from two rats given the same treatment and sampled at the same time were combined so that there were 25 samples from the sublingual group (10 rats/2×5 samples) and 20 samples from the oral group (8 rats/2×5 samples). Plasma was separated by centrifugation at 770 g for 10 minutes, placed in polypropylene tubes, frozen at −80° C., then shipped on dry ice to an outside laboratory to assay DCS levels. The results are set forth in Table 1, which shows the plasma levels of DCS (μg/ml) for the oral and sublingual doses as a function of the elapsed time since DCS administration. At the dosages studied, the resulting DCS plasma levels were near the lower detection limits of the assay.

TABLE 1

| Time Elapsed | Oral Formulation | Sublingual Formulation |
|---|---|---|
| (minutes) | DCS plasma level (μg/mL) | DCS plasma level (μg/mL) |
| 0 | 0 | 0 |
| 10 | 1.6 | 0.9 |
| 20 | 2.3 | 1.6 |
| 40 | 5.4 | 4.7 |
| 80 | 5.4 | 5.2 |

The results demonstrate that D-cycloserine can be administered sublingually to provide blood plasma concentrations comparable to those achieved by oral administration.

### Incorporation by Reference

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. Contemplated equivalents of the methods of treating anxiety related disorders disclosed here include administering fast acting compositions which otherwise correspond thereto, and which have the same general properties thereof, wherein one or more simple variations of substituents or components are made which do not adversely affect the characteristics of the methods and compositions of interest. The full scope of the invention should be determined by reference to the claims, along with their full scope of equivalents, and the specification, along with such variations.

The articles “a” and “an” are used herein to refer to one or to more than one (i.e. to at least one) of the grammatical object of the article. By way of example, “an element” means one element or more than one element.

Any ranges cited herein are inclusive, e.g., “between about 50 mg and 100 mg” includes compositions of 50 mg and 100 mg.

1.-26. (canceled)

27. A pharmaceutical composition suitable for sublingual administration comprising about 10 mg to about 80 mg D-cycloserine, wherein upon administration to a patient, the patient achieves a therapeutically effective serum concentration of D-cycloserine within forty minutes of administration.

28. The composition of claim 27, further comprising at least one pH-adjusting substance.

29. The composition of claim 27, further comprising an additive selected from the group consisting of: a glidant, a lubricant, a binder, a sweetener, a flavoring, and a coloring agent.

30. The composition of claim 27, further comprising at least one effervescent agent.

31. The composition of claim 27, wherein the composition is a solid.

32. The composition of claim 31, wherein the composition is a solid form selected from the group consisting of a tablet and a wafer.

33. The composition of claim 27, wherein the composition is in the form of a gel.

34. The composition of claim 27, wherein the composition is in the form of a spray.

35. The composition of claim 27, wherein the patient achieves a therapeutically effective serum concentration of D-cycloserine within twenty minutes of administration.

36. A method of treating an anxiety-related disorder in a subject in need thereof, the method comprising administering sublingually a therapeutically effective amount of D-cycloserine such that the patient achieves a therapeutically effective serum concentration of D-cycloserine within forty minutes of the administration.

37. The method of claim 36, wherein the patient achieves a therapeutically effective serum concentration of D-cycloserine within twenty minutes of the administration.

38. The method of claim 36, further comprising administering extinction training.

39. The method of claim 36, wherein the extinction training is administered within one-half hour of administering D-cycloserine.

40. The method of claim 36, further comprising administering an additional agent.

41. The method of claim 40, wherein the additional agent is a PDE-5 inhibitor selected from the group consisting of sildenafil, vardenafil, tadalafil, and udenafil.

42. The method of claim 36, wherein the anxiety-related disorder is an anxiety disorder.

43. The method of claim 36, wherein the anxiety-related disorder is selected from the group consisting of a mood disorder, an addictive disorder, an eating disorder, a movement disorder, erectile dysfunction, insomnia, and chronic pain.

44. The method of claim 36, wherein the D-cycloserine is administered before administration of the extinction training.

45. The method of claim 36, wherein the D-cycloserine is administered after administration of the extinction training.

46. The method of claim 36, wherein the D-cycloserine is administered during administration of the extinction training.

47. An improved method of treating an anxiety-related disorder by extinction training in a subject in need thereof, wherein the improvement comprises sublingually administering a therapeutically effective amount of D-cycloserine to the subject prior to completion of the extinction training.
